(19) United States
(12) Patent Application Publication
(10) Pub. No.: US 2021/0220609 A1
(43) Pub. Date: Jul. 22, 2021

(54) Implantable Pressure Sensor Probe and Catheter for Implanting a Pressure Sensor Probe

(71) Applicant: BIOTRONIK SE & Co. KG, Berlin (DE)

(72) Inventors: Andreas Arndt, Berlin (DE); Georg Nollert, Strasslach (DE)

(21) Appl. No.: 17/147,091

(22) Filed: Jan. 12, 2021

(30) Foreign Application Priority Data
Jan. 16, 2020 (EP) 20152121.8

(51) Int. Cl. A61M 25/01; A61M 25/06

(52) U.S. Cl. CPC A61M 25/0125; A61M 2025/0002; A61M 25/0662

(57) ABSTRACT

The disclosure relates to an implantable pressure sensor probe for measuring a pressure in the left atrium of a human heart, wherein a distal portion of the pressure sensor probe has a pressure sensor for detecting the pressure. A catheter is also described.

### CROSS-REFERENCE TO RELATED APPLICATIONS

This patent application claims the benefit of and priority to co-pending European Patent Application No. EP 20152121.8, filed on Jan. 16, 2020 in the European Patent Office, which is hereby incorporated by reference in its entirety.

### TECHNICAL FIELD

The disclosure relates to a method for implanting a pressure sensor into the left atrium of a human heart. In addition, the disclosure relates to an implantable pressure sensor probe for measuring a pressure in the left atrium of a human heart, and to a catheter for implanting a pressure sensor probe into the left atrium of a human heart.

### BACKGROUND

Such pressure sensors or pressure sensor probes, which measure a pressure in the left atrium, may be used, for example, to measure the filling pressure of the left heart in heart failure patients in order to optimise a drug therapy. Implantable pressure sensors for measuring pulmonary arterial pressure are known. However, these do not directly measure the pulmonary venous pressure, which is a decisive influencing variable for pulmonary congestion as a result of left heart failure, but instead measure a pressure in the pulmonary arterial system. The pulmonary arterial pressure follows the pulmonary venous pressure, but is also influenced at the same time by the transpulmonary gradient. It is not possible to clearly separate the two influences from each other, and therefore with this method a measurement inaccuracy remains with regard to the pulmonary venous pressure.

Transseptally implanted left atrial pressure sensors circumvent this disadvantage because they measure pulmonary venous pressure directly in the atrial diastole. The use of left atrial pressure sensors is known per se. For example, U.S. Pat. No. 8,016,764 describes the use of left atrial pressure sensors to determine ventricular dyssynchrony, depending on which pacemaker system or implantable cardioverter defibrillator (ICD) is controlled. U.S. Pat. No. 8,343,059 proposes to use a pressure sensor implanted in the left atrium to analyse a left atrial pressure and to perform a therapy depending on it.

Traditionally, left atrial pressure sensors are implanted through the atrial septum (i.e. transseptally) from the right atrium into the left atrium. However, such an implantation method is risky and is therefore reserved for experienced implanters. Complications with transseptal puncture have been reported in clinical trials (for example LAPTOP-HF). Furthermore, there is a risk that the pressure sensor will become overgrown with myocardium in the long-term, which may lead to a falsification of the sensor signal.

The present invention is directed toward overcoming one or more of the above-mentioned problems, though not necessarily limited to embodiments that do.

### SUMMARY

An object of the present invention is to overcome the presented disadvantages of known solutions in connection with implantable pressure sensors.

According to one aspect, a method for implanting a pressure sensor into the left atrium of a human heart is provided. Accordingly, the pressure sensor is inserted transvenously through the coronary sinus of the heart into the left atrium. This allows the pressure in the left atrium to be measured directly, but avoids a transseptal puncture when implanting the pressure sensor. Instead, the pressure sensor is implanted via the coronary sinus at low risk.

The proposed solution combines the advantages of a left atrial pressure sensor with the known technology of coronary sinus probes. For example, probes for cardiac resynchronisation therapy (CRT) that are implanted through the coronary sinus are known. However, conventional left ventricular CRT probes implanted through the coronary sinus do not have a pressure sensor that could be used to optimise the therapy of CRT patients.

For example, the pressure sensor may be mounted in a distal portion of a pressure sensor probe. In this case the pressure sensor probe may be formed, for example, according to a second aspect, which is explained in more detail below. The pressure sensor probe may be inserted into the coronary sinus via a suitable venous access, such as the subclavian vein. The coronary sinus runs through the left atrioventricular sulcus and thus shares a wall with the left atrium. By puncturing this wall, the pressure sensor may be permanently inserted into the left atrium at the distal portion of the pressure sensor probe.

Accordingly, the proposed implantation method according to one embodiment may comprise the following steps: providing an implantable pressure sensor probe comprising the pressure sensor, wherein the pressure sensor is arranged in a distal portion of the pressure sensor probe; puncturing the shared wall of the coronary sinus and the left atrium to create an opening in the wall; and transvenously implanting the pressure sensor probe into the coronary sinus in such a way that the distal portion with the pressure sensor projects through the opening into the left atrium.

For example, the pressure sensor may be inserted completely into the left atrium in this way in order to measure a filling pressure there. Preferably, the pressure sensor projects freely into the cavity of the left atrium so that myocardial cells are unable to grow over the pressure sensor.

During implantation, the coronary sinus (CS) is first probed with a suitable implantation tool. For example, a sheath of the type used for implanting CS electrodes of systems for cardiac resynchronisation therapy (CRT) is suitable for this. Advantageously, combinations of inner and outer catheters with a guide wire and dilator are used. The inner catheter may be bent at the distal end for puncturing the vessel wall in the left sulcus atrioventricularis, so that the opening of the distal end points towards the left atrium.

In order to assess the orientation of the tip of the catheter under fluoroscopy, it is useful to provide the tip of the catheter with one or more markers that are detectable by X-rays. For example, a cranial marker, a caudal marker and a posterior marker may be applied to the catheter. By rotating the catheter together with the markers, the correct position for the puncture may then be verified under fluoroscopy.

The shared wall between the coronary sinus and the left atrium may then be punctured for example using a puncture wire, and the dilator and finally the internal catheter may then be inserted. After removing the puncture wire and the dilator, the pressure sensor probe may be pushed through the implantation tool so that the distal portion of the pressure sensor probe with the pressure sensor protrudes into the left atrium. The implantation catheter may now be removed, and the implantation catheter may be slit and divided, for example as is common with CS systems.

The pressure sensor probe may be designed such that it anchors into the coronary sinus and the position of the pressure sensor in the left atrium remains stable. For example, the pressure sensor probe may have an anchoring portion for this purpose. The anchoring portion may comprise at least one element selected from the following group: barb, umbrella, plate, loop and flange.

According to an exemplary variant, to create the opening (i.e. the puncture)—similarly to a septal puncture—a puncture needle in a needle catheter is guided through the splinting guide lumen of a guide catheter to the posterior wall of the left atrium. Possible embodiments of a catheter usable as such a guide catheter are explained further below in connection with the third aspect. The puncture needle then punctures the posterior wall and the needle catheter is inserted into the left atrium. The puncture needle is then withdrawn completely and the pressure sensor arranged on the distal portion of the pressure sensor probe is inserted into the left atrium via the horizontal needle catheter. After fixation of the pressure sensor, the needle catheter is withdrawn.

In one embodiment, the pressure sensor is guided through a guide lumen formed in a catheter into the coronary sinus and then exits the guide lumen through a lateral exit opening. A catheter according to the third aspect may be used for this purpose, as will be discussed in more detail below. In line with this, for example, a catheter in the form of a specially adapted coronary sinus probe with stimulation electrodes for a CRT system may be used, which is provided with a guide lumen from a proximal end to just before a distal end. The guide lumen is large enough to accommodate the pressure sensor probe. In an exemplary embodiment, the coronary sinus probe tapers shortly before the distal end and has a lateral exit opening at this transition. Through the exit opening, the distal portion of the pressure sensor probe together with the pressure sensor may leave the coronary sinus probe and project through the puncture site into the left atrium.

The coronary sinus probe, similarly to the catheter described above, may be provided with markers so that the position of the exit opening may be adjusted under fluoroscopy in the direction of the left atrium. The coronary sinus probe may then be placed in the coronary sinus in such a way that the stimulation electrodes lie in a suitable venous vessel and the exit opening points in the direction of the left atrium. A puncture wire may then be used to puncture the shared wall between the coronary sinus and the left atrium and the pressure sensor probe may be inserted, after which the wire is removed. For example, the pressure sensor probe may be provided with a lumen for the puncture wire.

In another variant of the implantation method, a balloon of the catheter is filled in the coronary sinus to fix a position of the exit opening. For example, in an exemplary embodiment of that variant, a splint is used in the coronary sinus similarly to the coronary sinus probe described above, with the difference that no stimulation electrode is used, but instead a balloon catheter placed specifically for this purpose. The balloon catheter may have a balloon at its distal end that may be filled and deflated. Furthermore, as described above with reference to the coronary sinus probe, the balloon catheter may have a guide lumen with a lateral exit opening for the pressure sensor probe, the exit opening being located just before the distal end of the balloon catheter. The balloon catheter is advanced into the coronary sinus with the balloon deflated. The balloon is then filled, thus wedging the balloon catheter into the coronary sinus from the inside. This fixes the exit point relative to the wall between the coronary sinus and the left atrium in a position suitable for puncture. Immediately afterwards, the wall is punctured through the exit opening and the pressure sensor probe is pushed in. Lastly, the balloon is deflated again to release the blood flow in the venous system.

It is also within the scope of the disclosure that a proximal portion of the pressure sensor probe may be connected to an implanted sensing device designed to receive signals indicative of a pressure sensed by the pressure sensor in the left atrium. For example, a proximal end of the pressure sensor probe may leave the venous vascular system at a suitable location, for example in the subclavian vein, and may be connected to a preferably pectorally implanted unit which detects the pressure signals from the sensor, stores them if necessary, processes them, and sends them to a device located outside the body. This unit may be a special unit for measuring the pressure signals only. However, it may also be, for example, a one-, two- or three-chamber pacemaker or an implantable cardioverter defibrillator (ICD) that is set up for the connection of a pressure sensor probe.

According to a second aspect, an implantable pressure sensor probe for measuring a pressure in the left atrium of a human heart is disclosed. In this regard it is provided that a distal portion of the pressure sensor probe comprises a pressure sensor for sensing the pressure.

The distal portion of the pressure sensor probe may further comprise an anchoring portion for anchoring the distal portion in tissue.

The pressure sensor probe or pressure sensor may be used in the method disclosed herein. Accordingly, what has been explained above with respect to pressure sensor probes and pressure sensors in connection with the implantation method applies analogously to some embodiments of the pressure sensor probe or pressure sensor, and vice versa.

The anchoring portion may be used to anchor the pressure sensor probe in the tissue, for example in the implanted state, in such a way that the position of the pressure sensor in the left atrium remains stable. The anchoring portion may comprise at least one element selected from the following group: barb, umbrella, plate, loop and flange.

According to a refinement, the pressure sensor probe may also have at least one electrode, for example a stimulation electrode. For example, this may be an electrode (or plurality of electrodes) of an implantable cardioverter defibrillator (ICD) or an electrode for cardiac resynchronisation therapy (CRT).

In principle, the pressure sensor probe either may be designed as a stand-alone implant or may be integrated as an additional component in an implantable system. For example, the pressure sensor probe may be integrated into a coronary sinus probe of a CRT system (or another type of pacemaker system) or may be combined as an additional probe with single- or dual-chamber pacemakers or ICD systems. In combination with CRT systems, the pressure sensor signal obtained in this way may be used, for example, to optimize therapy. Single-, dual- or triple-chamber pacemakers or ICD systems may also be advantageously expanded in this way to include the possibility of left atrial pressure measurement. Such systems, which additionally comprise a pressure sensor probe, therefore also lie within the scope of the invention.

A third aspect relates to a catheter for implanting a pressure sensor probe into the left atrium of a human heart. The catheter comprises a catheter body in which a guide lumen is formed for receiving/guiding the pressure sensor probe. A distal portion of the catheter body has the following: a lateral exit opening for guiding a distal portion of the pressure sensor probe out of the guide lumen; and a marker detectable by X-rays to determine a location of the exit opening.

The catheter may be used in the method disclosed herein. Further, the catheter may be provided and designed for implanting a pressure sensor probe disclosed herein. Accordingly, what is described above with respect to pressure sensor probes and catheters applies analogously to some embodiments of the catheter or pressure sensor probe, and vice versa. Features and embodiments of the various aspects may be combined as desired.

For example, as described above in connection with the implantation method, a balloon may be arranged on the distal portion of the catheter body. In other words, the catheter may be designed as a balloon catheter. This makes it possible to wedge the balloon catheter in a suitable position from the inside in the coronary sinus, as explained above. For example, this allows the exit point to be fixed in a suitable position relative to the wall between the coronary sinus and the left atrium.

As already explained, the marker may be used to rotate the catheter under fluoroscopy in such a targeted manner that the exit opening points in the direction of the left atrium (and there more precisely to a point intended for puncturing). The marker may, for example, comprise a heavy metal that is impermeable to X-rays and therefore easily recognisable in fluoroscopy. In one variant, the marker is T-shaped. This makes it easy to read the orientation of the catheter body under fluoroscopy.

Additional features, aspects, objects, advantages, and possible applications of the present invention will become apparent from a study of the exemplary embodiments and examples described below, in combination with the Figures and the appended claims.

### BRIEF DESCRIPTION OF THE DRAWINGS

Further advantages and embodiments are described below with reference to the figures, in which:

FIG. 1 shows an exemplary and schematic flow chart of a method for implanting a pressure sensor into the left atrium of a human heart;

FIG. 2 shows a distal portion of an implantable pressure sensor probe according to an exemplary embodiment;

FIG. 3 shows a schematic rear view of a human heart with a pressure sensor probe extending into the coronary sinus;

FIG. 4 shows a schematic internal view of the left atrium of the heart from FIG. 3, with a distal portion of the pressure sensor probe with a pressure sensor extending through a puncture point into the left atrium;

FIG. 5 shows a catheter inserted into a coronary sinus for implanting a pressure sensor probe according to an exemplary embodiment; and

FIG. 6 shows another exemplary embodiment of a catheter, wherein the catheter has an inflatable balloon distally.

### DETAILED DESCRIPTION

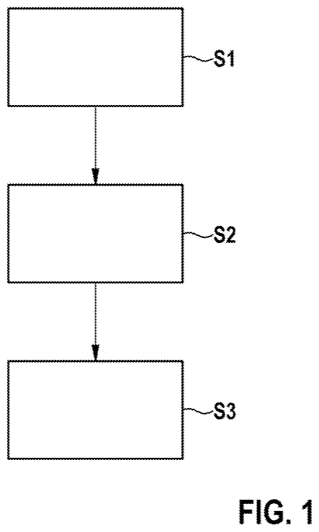

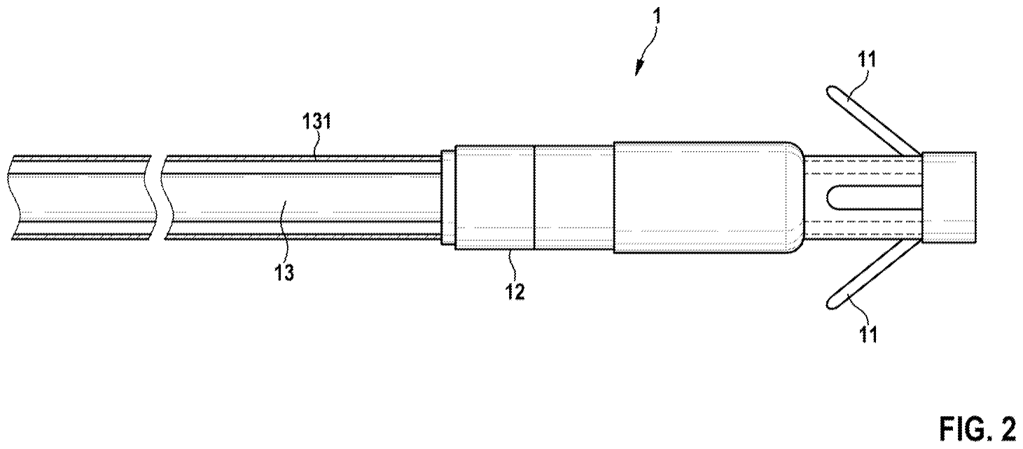

FIG. 1 shows a schematic flow chart of a method for implanting a pressure sensor 12 into the left atrium LA of a human heart according to an exemplary embodiment. The pressure sensor 12 is implanted transvenously (for example via the subclavian vein) through the coronary sinus CS of the heart into the left atrium LA. In the following explanation of the method, reference is also made immediately to FIGS. 2 to 6, which show views of the left atrium LA or views of components relevant for implantation, such as a catheter 2 and a pressure sensor probe 1, in greater detail.

In a first step S1, the method according to FIG. 1 comprises providing an implantable pressure probe 1 which has the pressure sensor 12 to be implanted. For example, a probe similar to the known coronary sinus probes of CRT systems may be additionally equipped with a pressure sensor 12 at its distal end.

FIG. 2 shows a distal portion of an implantable pressure sensor probe 1 for measuring a pressure in the left atrium LA according to an exemplary embodiment. The pressure sensor 12 is arranged on the distal portion of the pressure sensor probe 1. A cable 13 leads to the pressure sensor 12 and is electrically insulated from the surrounding tissue by an insulation 131. For example, signals indicating a pressure detected by the pressure sensor 12 in the left atrium LA may be supplied to an implanted or extracorporeal receiving device via the cable 13 of the pressure sensor probe 1.

In the embodiment according to FIG. 2, the distal portion of the pressure sensor probe 1 comprises, in addition to the one pressure sensor 12, an anchoring portion 11 for anchoring the distal portion of the pressure sensor probe 1 in the tissue. The anchoring portion 11, which in this case is in the form of a number of barbs, may anchor the pressure sensor probe 1 in the tissue, for example in the implanted state, in such a way that the position of the pressure sensor 12 in the left atrium LA remains stable.

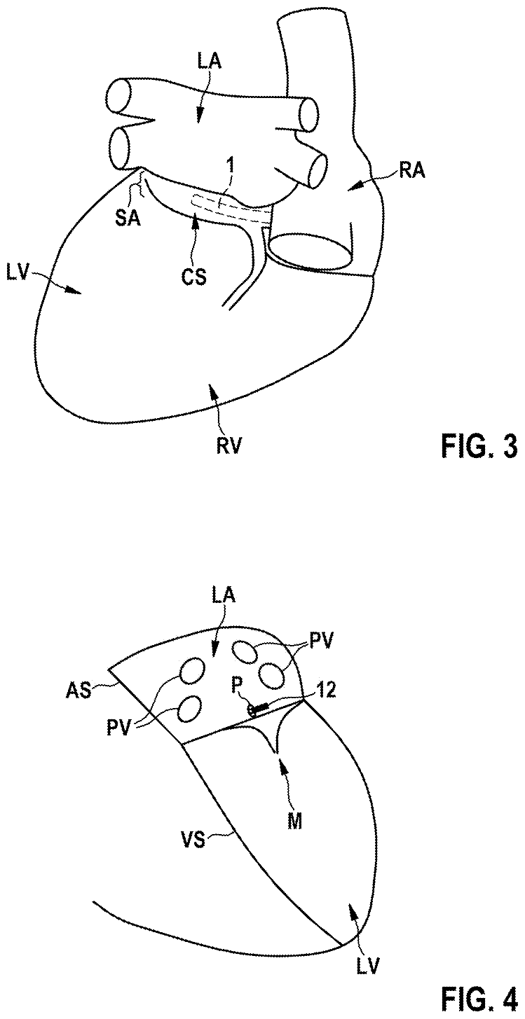

In a second step S2 of the exemplary embodiment of the implantation method according to FIG. 1, the shared wall of the coronary sinus CS and the left atrium LA is punctured to create an opening P in that wall. For orientation regarding the heart anatomy, please refer to FIGS. 3 and 4.

FIG. 3 shows a schematic rear view of a human heart, with the right atrium RA and left atrium LA located above the right ventricle RV and above the left ventricle LV, respectively. The coronary sinus CS together with the atrioventricular sulcus SA are also shown. The coronary sinus CS extends along a posterior wall of the left atrium, which is punctured in the second method step S2.

FIG. 4 shows a schematic internal view of the left atrium LA of the heart from FIG. 3. The attachment points of the pulmonary veins PV and the mitral valve M between the left atrium LA and the left ventricle LV are visible. The atrial septum AS and the ventricular septum VS are also schematically drawn. In the vicinity of the mitral valve M, the wall of the left atrium LA has the opening (puncture point) P that was created in the second method step S2 from the coronary sinus CS.

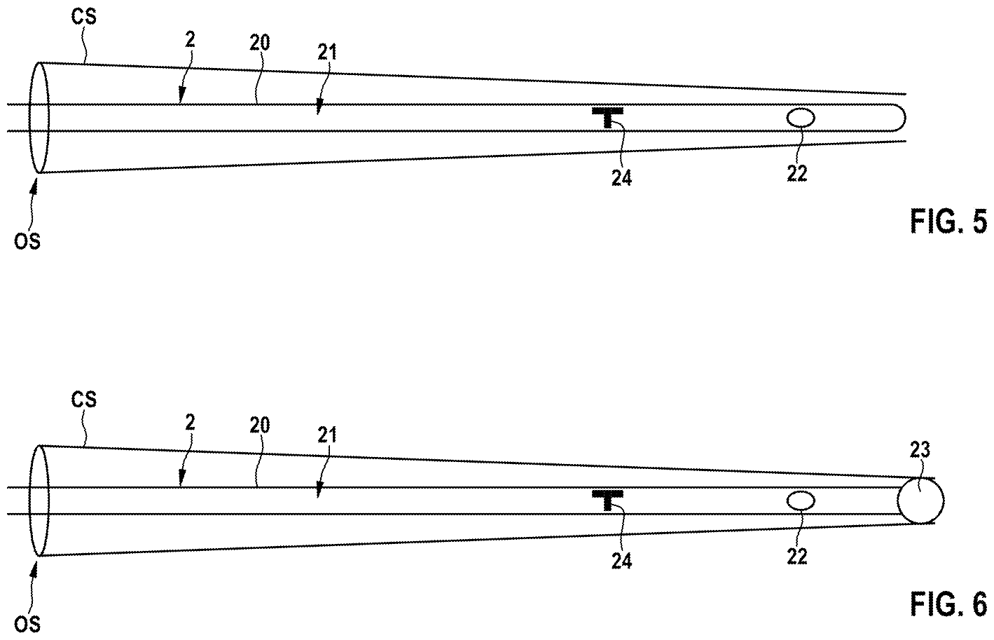

According to an exemplary variant, to create the opening P—similarly to a septal puncture—a puncture needle in a needle catheter is guided through the splinting guide lumen of a guide catheter to the posterior wall of the left atrium LA. Possible embodiments of a catheter 2 that may be used as such a guide catheter are shown in FIGS. 5 and 6 and are explained in more detail below.

The puncture needle then punctures the posterior wall and the needle catheter is inserted into the left atrium LA. The puncture needle is then withdrawn completely and the pressure sensor 12 arranged on the distal portion of the pressure sensor probe 1 is inserted into the left atrium LA via the horizontal needle catheter. After fixation of the pressure sensor 12, the needle catheter is withdrawn.

In a second step S2 of the exemplary embodiment of the implantation method according to FIG. 1, the pressure probe 1 is implanted transvenously (for example via the subclavian vein) into the coronary sinus CS, moreover in such a way that the distal portion with the pressure sensor 12 projects through the opening P into the left atrium LA.

This is illustrated in more detail in FIGS. 3 and 4. FIG. 3 shows a portion of a pressure sensor probe 1 implanted in the coronary sinus CS in the manner described. FIG. 4 shows a distal portion of the pressure sensor probe 1 from FIG. 3 from an internal perspective of the left atrium LA, said distal portion extending, with the pressure sensor 12 arranged on it, through the puncture point P into the left atrium LA.

In FIG. 4 it is clearly visible that the pressure sensor 12 in this embodiment is located completely in the left atrium LA and may thus directly measure a filling pressure prevailing there. In this case, the pressure sensor 12 projects freely into the cavity of the left atrium LA. This has the advantage that myocardial cells are unable to grow over the pressure sensor.

In the following, exemplary embodiments of a catheter 2 for implanting a pressure sensor probe 1 into the left atrium LA are explained with reference to FIGS. 5 and 6. The catheter is inserted into a distally tapering coronary sinus CS. An ostium OS of the coronary sinus is shown at the left edge of each image.

The catheter 2 shown in FIG. 5 comprises a tubular catheter body 20, in which a guide lumen 21 is formed for the pressure sensor probe 1 to be implanted. The guide lumen 21 is large enough to accommodate the pressure sensor probe 1. A distal portion of the catheter body 20 has a lateral exit opening 22 for guiding the distal portion of the pressure sensor probe 1 out of the guide lumen 21. The pressure sensor 12 arranged on the distal portion of the pressure sensor probe 1 may thus be guided through the guide lumen 21 into the coronary sinus CS, then leave the guide lumen 21 through the lateral exit opening 22 and project through the puncture point P into the left atrium LA.

In addition, a marker 24 is attached to the distal portion of the catheter body 20 and is detectable by X-rays in order to determine a position of the exit opening 22. In particular, the position of the outlet opening 22 in the direction of the left atrium LA may be adjusted under fluoroscopy by selective rotation of the catheter body 20. The marker 24 may, for example, comprise a heavy metal that is impermeable to X-rays and therefore easily recognisable by fluoroscopy.

In the shown variant, the marker 24 is T-shaped. This makes it easy to read an orientation of the catheter body 22 (and in particular a direction of the exit opening 22) under fluoroscopy. In this case, the marker 24 is in the form of an upright T on the same semicircle of the catheter body 20 as the exit opening 22. If the marker 24 shows an upright T in fluoroscopy, the exit opening 22 is directed ventrally (this corresponds to a correct rotation for access to the left atrium LA). On the other hand, if the T is inverted, the exit opening points dorsally and must be rotated 180°.

In the present exemplary embodiment, there is only a single marker 24 on the catheter body 20. Additional markers may in principle facilitate orientation.

The variation of the catheter 2 from FIG. 5 shown in FIG. 6 additionally has a balloon 23 distally, which may be filled and deflated. In other words, the catheter 2 is designed as a balloon catheter. This makes it possible to temporarily wedge the balloon catheter 2 in a suitable position from the inside in the coronary sinus CS during implantation by filling the balloon 23. For example, this allows the exit point 22 of the catheter body 20 to be fixed in a suitable position relative to the wall between the coronary sinus CS and the left atrium LA for puncture. As described above, the catheter body 20 may first be deliberately rotated into the suitable position by means of the marker 24 under fluoroscopy.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE SIGNS

1 Pressure sensor probe
11 Anchoring portion
12 Pressure sensor
13 Connecting cable
131 Insulation
2 Catheter
20 Catheter body
21 Guide lumen
22 Exit opening
23 Balloon
24 Marker
AS Atrial septum
CS Coronary sinus
LA Left atrium
LV Left ventricle
M Mitral valve
OS Ostium of the coronary sinus
P Opening (puncture point)
PV Pulmonary veins
RA Right atrium
RV Right ventricle
SA Atrioventricular sulcus
S1-S3 Method steps
VS Chamber septum

What is claimed is:

1. A method for implanting a pressure sensor into a left atrium of a human heart, comprising the steps of
inserting the pressure sensor transvenously through a coronary sinus of the heart into the left atrium.

2. The method according to claim 1, further comprising the steps of:
providing an implantable pressure sensor probe comprising the pressure sensor, wherein the pressure sensor is arranged in a distal portion of the pressure sensor probe;
puncturing a shared wall of the coronary sinus and the left atrium to create an opening in the shared wall; and
transvenously implanting the pressure sensor probe into the coronary sinus in such a way that the distal portion with the pressure sensor projects through the opening into the left atrium.

3. The method according to claim 2, wherein the pressure sensor probe is inserted into the coronary sinus via a suitable venous access through the subclavian vein.

4. The method according to claim 1, further comprising the steps of:
probing the coronary sinus with a suitable implantation tool using combinations of inner and outer catheters with a guide wire and a dilator, wherein the inner catheter is bent at the distal end for puncturing the vessel wall in a left sulcus atrioventricularis, so that the opening of the distal end of the inner catheter points towards the left atrium.

5. The method according to claim 4, wherein an orientation of the tip of the inner catheter is assessed under fluoroscopy using with one or more markers that are detectable by X-rays arranged at the tip of the inner catheter, preferably a cranial marker, a caudal marker and a posterior marker are applied to the inner catheter

6. The method according to claim 1, further comprising the steps of:
puncturing a shared wall between the coronary sinus and the left atrium using a puncture wire;
inserting a dilator and finally an internal catheter;
removing the puncture wire and the dilator;
pushing the pressure sensor probe through the implantation tool so that the distal portion of the pressure sensor probe with the pressure sensor protruding into the left atrium; and
removing the internal catheter.

7. The method according to claim 2, wherein a puncture needle in a needle catheter is used to create the opening, wherein the puncture needle in the needle catheter is guided wo through the splinting guide lumen of a guide catheter to the posterior wall of the left atrium.

8. The method according to claim 7, further comprising the following steps:
puncturing, using the puncture needle, a posterior wall;
inserting the needle catheter into the left atrium;
withdrawing the puncture needle completely;
inserting the pressure sensor, arranged on the distal portion of the pressure sensor probe, into the left atrium via the needle catheter; and
withdrawing the needle catheter after the fixation of the pressure sensor.

9. The method according to claim 1, wherein the distal portion of the pressure sensor probe has an anchoring portion for anchoring the distal portion in tissue.

10. The method according to claim 9, wherein the anchoring portion comprises at least one element selected from the following group: barb, umbrella, plate, loop and flange.

11. The method according to claim 1, wherein a catheter for implanting a pressure sensor probe into the left atrium (LA) of a human heart is used, wherein the catheter comprises a catheter body in which a guide lumen for the pressure sensor probe is formed, wherein a distal portion of the catheter body comprises:
a lateral exit opening for leading a distal portion of the pressure sensor probe out of the guide lumen; and
a marker detectable by X-rays to determine a position of the exit opening.

12. The method according to claim 11, further comprising the steps of:
arranging a balloon on the distal portion of the catheter body.

13. The method according to claim 11, wherein the marker comprises a heavy metal.

14. The method according to one of claim 11, wherein the marker is T-shaped.

15. The method according to claim 1, further comprising the steps of:
providing an implantable pressure sensor probe comprising the pressure sensor, wherein the pressure sensor is arranged in a distal portion of the pressure sensor probe;
providing a balloon catheter having a guide lumen with a lateral exit opening for the pressure sensor probe;
advancing the balloon catheter into the coronary sinus with the balloon deflated;
wedging the balloon catheter into the coronary sinus from an inside and fixing an exit point relative to a shared wall between the coronary sinus and the left atrium in a position suitable for puncture;
puncturing the shared wall through the exit opening;
pushing the pressure sensor probe through the guide lumen so that the distal portion of the pressure sensor probe with the pressure sensor protruding into the left atrium; and
deflating the balloon.

16. The method according to claim 1, further comprising the steps of:
measuring, by the pressure sensor, a pressure in the left atrium and generating pressure signals;
detecting, by a pectorally implanted unit, the pressure signals from the pressure sensor; and
sending, by the pectorally implanted unit, the detected pressure signals to a device located outside the human body.
